(19) United States
(12) Patent Application Publication
(10) Pub. No.: US 2022/0096717 A1
(43) Pub. Date: Mar. 31, 2022

(54) AXITINIB-LOADED NANOFIBER MEMBRANE, PREPARATION METHOD FOR THE SAME, AND ITS USE OF ANTI-ADHESION AFTER A SURGERY

(71) Applicant: SHANGHAI CHILDREN'S MEDICAL CENTER, SHANGHAI JIAO TONG UNIVERSITY SCHOOL OF MEDICINE, Shanghai (CN)

(72) Inventors: Hao ZHANG, Shanghai (CN); Bei FENG, Shanghai (CN); Xingang WANG, Shanghai (CN); Zihao DAI, Shanghai (CN); Yiwei LIU, Shanghai (CN); Lincai YE, Shanghai (CN)

(73) Assignee: SHANGHAI CHILDREN'S MEDICAL CENTER, SHANGHAI JIAO TONG UNIVERSITY SCHOOL OF MEDICINE

(21) Appl. No.: 17/136,630

(22) Filed: Dec. 29, 2020

(30) Foreign Application Priority Data
Sep. 30, 2020 (CN) 202011055640.4

(51) Int. Cl. A61L 31/16; A61K 9/48; A61L 31/04; A61K 31/4439; A61P 41/00

(52) U.S. Cl. CPC A61L 31/16; A61K 9/4825; A61L 31/041; A61L 2400/12; A61P 41/00; A61L 2300/424; A61L 2300/204; A61K 31/4439

(57) ABSTRACT

The application discloses a use of nanofiber membrane in the preparation of medical apparatus for anti-adhesion after a surgery, wherein small molecule drug, inhibiting vascular endothelial growth factor and/or inhibiting vascular endothelial growth factor receptor, is loaded into the nanofiber membrane. The nanofiber membrane provided in the present application has the following advantages: it has good biocompatibility, excellent mechanical properties, excellent flexibility and smoothness when exposed to water, good air permeability, and has the ability to effectively prevent adhesion between the heart and surrounding tissues. In addition, it can also be applied to other surgical operations, such as prevention of adhesions in the abdominal cavity, pelvis, and tendons.

### BACKGROUND OF THE INVENTION

### 1. Field of the Invention

The invention relates to the field of biomedical materials, and more particularly, to an Axitinib-loaded nanofiber membrane, preparation method for the same, and its use of anti-adhesion after a surgery.

### 2. Description of the Related Art

Adhesion is one of the common complications after surgery and may lead to serious consequences. For example, pelvic and abdominal adhesions can cause intestinal obstruction, pelvic or abdominal pain, infertility in women and other diseases. In most cases, patients who have undergone cardiac surgery will need another surgery or even multiple surgeries. Since the original anatomical levels and gaps disappear due to the presence of adhesions developed in the first cardiac surgery, a further thoracotomy operation can be a daunting task and full of risks. Preventing adhesions after cardiac surgery can greatly reduce the difficulty of second heart operation, reduce intraoperative and postoperative bleeding, reduce operation time and costs, and even reduce mortality.

“Physical barrier” method is most commonly used to prevent adhesion after cardiac surgery. The method is to place barrier materials between the wound and the surrounding tissues for effecting physical separation therebetween, so as to reduce adhesions. In such materials, expanded polytetrafluoroethylene (ePTFE) is a material for prevention of adhesion in the heart, which has always been a focus of the research in the world. However, the expanded polytetrafluoroethylene (ePTFE) is a non-degradable membrane material. Therefore, if ePTFE remains in the body for a long period of time, mechanical irritation and foreign body reaction may occur. And long-term foreign body reaction may cause infections. In recent years, medical experts and materials experts have focused their attention on biodegradable materials. After implanted in the organism, such biodegradable materials will gradually degrade in the physiological environment and be metabolized and absorbed by the body. In this way, it eliminates the need for a second operation of removing the biodegradable materials from the body.

In addition to the “physical barrier” method, the “drug prevention” method is also a strategy for the prevention of post-surgical adhesions. The “drug prevention” method is mainly to use drugs to reduce inflammatory response of the tissues and exudation, and to promote fibrinolysis. Systemic administration can lead to many side effects and its effectiveness has not been well defined; and topical administration has some disadvantages that drugs administered to the patient are easy to loss, and efficacy will not sustain for a long period of time. Therefore, drug therapy is not widely used in this regard.

The drugs work in four different stages of adhesion formation: (1) cell exudate—inflammation—fibrin monomer formation (within 24 hours); (2) fibrin deposition—cellulose deposition—fibrinolysis and collagen deposition (24-72 hours); (3) entry of new blood vessels and lymphatic vessels into new connective tissues (1 week); (4) the adhesion continues to mature—dense fiber band formation (2 weeks). Corticosteroid anti-inflammatory drugs were used to prevent cardiac adhesions. The corticosteroid anti-inflammatory drugs mainly acted on the above-mentioned stage (1). However, it was found that such drugs have side effects, such as delaying healing time and increasing the probability of infection, so they are not widely used in the clinical applications. Further, topical use of plasminogen activator for stage (2) may promote fibrin degradation, however, it will cause intrapericardial hemorrhage. For those reasons, there are few reports about the clinical applications of plasminogen activator.

Axitinib is a new generation of anti-tumor drug developed by Pfizer. Clinically, it is mainly used for adult patients with advanced renal cell carcinoma (RCC) who have received failure of prior tyrosine kinase inhibitor or cytokine therapies. Axitinib has remarkable anti-angiogenic activity. It can selectively inhibit the activity of vascular endothelial growth factor (VEGF), and reduce angiogenesis, thereby inhibiting tumor growth. The 2012 international multi-center AXIS study showed that Axitinib was better than sorafenib (NCT00678392). The clinical researches carried out in China 2015 showed that Axitinib was better than sorafenib. Axitinib has excellent chemical structural stability. It is a small molecule compound (chemical formula: C₂₂H₁₈N₄OS, molecular weight: 386.47). By contrast, for macromolecular proteins, such as monoclonal antibodies, their spatial structure is easily damaged during the loading process, then they lose activity.

As a natural high polymer material, gelatin is non-toxic, immunogenic, and biodegradable. Polycaprolactone (PCL) is a synthetic high polymer material approved by the FDA (U.S. Food and Drug Administration). Products after the degradation of PCL are CO₂ and H₂O, and they are free from any toxic side effects.

### SUMMARY OF THE INVENTION

The application provides the use of a nanofiber membrane in the preparation of medical apparatus for anti-adhesion after a surgery.

The detailed technical solution is as follows:

a use of a nanofiber membrane in the preparation of medical apparatus for anti-adhesion after a surgery, wherein small molecule drug, inhibiting vascular endothelial growth factor and/or inhibiting vascular endothelial growth factor receptor, is loaded into the nanofiber membrane.

Furthermore, the surgery is cardiac surgery.

Furthermore, the anti-adhesion is to prevent adhesion between the heart to sternum and/or pericardium.

Furthermore, the small molecule drug is Axitinib.

Furthermore, the amount of Axitinib loaded in the nanofiber membrane is not less than 1%.

Furthermore, the amount of Axitinib loaded in the nanofiber membrane is in a range of 2% to 30%.

Furthermore, the nanofiber membrane is a gelatin/polycaprolactone nanofiber membrane.

Furthermore, the nanofiber membrane is obtained from a gelatin/polycaprolactone spinning solution containing Axitinib by electrospinning.

Furthermore, the nanofiber membrane is produced by a method comprising the steps of: preparing a first spinning solution and a second spinning solution, respectively, wherein the first spinning solution is gelatin/polycaprolactone spinning solution containing Axitinib, and the second spinning solution is gelatin/polycaprolactone spinning solution not containing Axitinib; extracting the first spinning solution, the second spinning solution in sequence, and the first spinning solution is implemented for electrospinning process; finally, obtaining a nanofiber membrane with a sandwich structure.

Furthermore, the nanofiber membrane is produced by a method comprising the steps of: preparing a core spinning solution and a shell spinning solution, respectively, wherein the core spinning solution contains Axitinib, and the shell spinning solution is gelatin/polycaprolactone spinning solution not containing Axitinib; performing coaxial electrospinning on the two spinning solution; and finally, obtaining a nanofiber membrane with a shell-core structure.

By adopting the above-mentioned technical solutions, the present invention has the beneficial effects that

the nanofiber membrane provided in the present application has good biocompatibility, excellent mechanical properties, excellent flexibility and smoothness when exposed to water, good air permeability, and has the ability to effectively prevent adhesion between the heart and surrounding tissues. In addition, for example, it can also be applied to other surgical operations, such as prevention of adhesions in the abdominal cavity, pelvis, and tendons.

The Axitinib used in this application is a clinical drug with high safety, stable structure, and it can be uniformly dispersed and wrapped in nanofiber materials.

The preparation method of the nanofiber membrane of the present application is simple in operation, and it is low in cost and less time-consuming. It is expected to realize continuous industrialized production of nanofibers. In addition, it is possible to produce a nanofiber membrane with different shapes and thicknesses, having controllable material degradation rates, and controllable drug release rates simply by adjusting various components and ratios thereof, and by appropriately changing the spinning conditions.

### BRIEF DESCRIPTION OF THE DRAWINGS

The accompanying drawings may facilitate the understanding of the application and form a part of the application. The exemplary embodiments of the present application, together with the description, serve to explain the principles of the present invention without limiting the scope of the application.

FIG. 1: pictures showing solubility of Axitinib. A is a picture in which Axitinib is dissolved in water and trifluoroethanol; B is a picture in which gelatin and polycaprolactone are dissolved in trifluoroethanol with and without the addition of Axitinib to the electrospinning solution.

FIG. 2: Scanning electron micrographs. A and B are scanning electron micrographs of gelatin/polycaprolactone nanofiber membranes (at different magnifications); C and D are scanning electron micrographs of Axitinib-loaded gelatin/polycaprolactone nanofiber membranes (at different magnifications).

FIG. 3: Cell experimental results. A and B show the Live & Dead cells of cells in the heart tissue are stained on the material (green: live cells, red: dead cells); C and D show EdU of endothelial cells are stained on the material (purple: proliferation, blue: cell nucleus); E and F show the nuclear of endothelial cells is stained on the material (blue: cell nucleus). Scale bar: 50 μm.

FIG. 4: animal experimental results. One month after surgery, A and B are general views of positive control groups; one month after surgery, C and D are general views of gelatin/polycaprolactone material group; one month after surgery, E and F are general views of Axitinib-loaded gelatin/polycaprolactone material group.

### DETAILED DESCRIPTION

The technical solution set forth in the embodiments of the present invention will now be described clearly and fully hereinafter with reference to the accompanying drawings of the embodiments of the present invention. Obviously, such embodiments provided in the present invention are only part of the embodiments instead of all embodiments. It should be understood that all the other embodiments obtained from the embodiments set forth in the present invention by one skilled in the art without any creative work fall within the scope of the present invention.

### Example 1

0.5 g of gelatin and 0.5 g of polycaprolactone were weighed and dissolved in 10 ml of trifluoroethanol. 30 microliters of acetic acid was added to the resulting solution. Stirring the resulting solution until it was well mixed. Then 10% g/ml by weight of spinning solution was prepared.

A 10 ml syringe and a syringe needle of 1.2 mm inner diameter were used to extract the spinning solution. The syringe containing the extracted spinning solution was fixed on an electrospinning device for electrospinning process. A drum was used as a receiving device to obtain the gelatin/polycaprolactone nanofiber membrane. Parameters involved in the electrospinning process were configured as follows: voltage 5-50 kv, receiving distance 5-30 cm, injection rate 0.5-10 ml/h, temperature 10-40° C., relative humidity 20-80%.

### Example 2

100 mg of Axitinib was weighed and dissolved in 10 ml of trifluoroethanol. 40 microliters of acetic acid was added to the resulting solution. Stirring the resulting solution until it was well mixed. 0.5 g of gelatin and 0.5 g of polycaprolactone were added to the mixed solution to obtain 10% g/ml by weight of spinning solution.

A 10 ml syringe and a syringe needle of 1.2 mm inner diameter were used to extract the spinning solution. The syringe containing the extracted spinning solution was fixed on an electrospinning device for electrospinning process. A drum was used as a receiving device to obtain a Axitinib-loaded gelatin/polycaprolactone nanofiber membrane. Parameters involved in the electrospinning process were configured as follows: voltage 5-50 kv, receiving distance 5-30 cm, injection rate 0.5-10 ml/h, temperature 10-40° C., relative humidity 20-80%.

### Example 3

Preparation of two different spinning solutions: (1) 100 mg of Axitinib was weighed and dissolved in 10 ml of trifluoroethanol. 40 microliters of acetic acid was added to the resulting solution. Stirring the resulting solution until it was well mixed. 0.5 g of gelatin and 0.5 g of polycaprolactone were added to the mixed solution to obtain 10% g/ml by weight of spinning solution; (2) 0.5 g of gelatin and 0.5 g of polycaprolactone were weighed and dissolved in 10 ml of trifluoroethanol. 30 microliters of acetic acid was added to the resulting solution. Stirring the resulting solution until it was well mixed. Then 10% g/ml by weight of spinning solution was prepared.

A 10 ml syringe and a syringe needle of 1.2 mm inner diameter were used to extract the first spinning solution. The syringe containing the extracted spinning solution was fixed on an electrospinning device for electrospinning process. A drum was used as a receiving device. After 1 ml of spinning solution was subjected to electrospinning process, the second spinning solution is extracted. Then when 1 ml of the second spinning solution was subjected to electrospinning process, 1 ml of the first spinning solution was extracted again, so as to obtain an Axitinib-loaded gelatin/polycaprolactone nanofiber membrane with a sandwich structure. Parameters involved in the electrospinning process were configured as follows: voltage 5-50 kv, receiving distance 5-30 cm, injection rate 0.5-10 ml/h, temperature 10-40° C., relative humidity 20-80%.

### Example 4

Preparation of two different spinning solutions: (1) 100 mg of Axitinib was weighed and dissolved in 5 ml of trifluoroethanol, and the resulting solution was stirred until it was well mixed; (2) 0.5 g of gelatin and 0.5 g of polycaprolactone were weighed and dissolved in 10 ml of trifluoroethanol. 30 microliters of acetic acid was added to the resulting solution. Stirring the resulting solution until it was well mixed. Then 10% g/ml by weight of spinning solution was prepared.

An Axitinib-loaded gelatin/polycaprolactone nanofiber membrane with a shell-core structure is prepared by coaxial electrospinning using the following method: selecting a drum as a receiving device, using the first spinning solution as core spinning solution, using the second spinning solution as shell spinning solution, then performing coaxial electrospinning on the two spinning solutions at a given supply rate. Parameters involved in the electrospinning process were configured as follows: voltage 5-50 kv, receiving distance 5-30 cm, injection rate 0.5-10 ml/h, temperature 10-40° C., relative humidity 20-80%.

### Example 5

Preparation of two different spinning solutions: (1) 100 mg of Axitinib was weighed and dissolved in 5 ml of trifluoroethanol, and the resulting solution was stirred until it was well mixed; (2) 0.5 g of gelatin and 0.5 g of polycaprolactone were weighed and dissolved in 10 ml of trifluoroethanol. 30 microliters of acetic acid was added to the resulting solution. Stirring the resulting solution until it was well mixed. Then 10% g/ml by weight of spinning solution was prepared.

Axitinib-loaded gelatin/polycaprolactone nanofiber membranes with a shell-core structure and a sandwich structure were prepared by coaxial electrospinning using the following method: selecting a drum as a receiving device, using the first spinning solution as core spinning solution, using the second spinning solution as shell spinning solution, then performing coaxial electrospinning on 1 ml of the two spinning solutions at a given supply rate. Then changing the spinning system. The second spinning solution was separately subjected to electrospinning on the initial drum, the receiving device, for spinning about 1 ml of the total volume; finally, using the first spinning solution as core spinning solution, using the second spinning solution as shell spinning solution, and spinning 1 ml of the total volume of the spinning solution by coaxial electrospinning Parameters involved in the electrospinning process were configured as follows: voltage 5-50 kv, receiving distance 5-30 cm, injection rate 0.5-10 ml/h, temperature 10-40° C., relative humidity 20-80%.

### Example 6

Preparation of three different spinning solutions: (1) 200 mg of Axitinib was weighed and dissolved in 5 ml of trifluoroethanol, and the resulting solution was stirred until it was well mixed. (2) 0.8 g of gelatin and 0.4 g of polycaprolactone were weighed and dissolved in 10 ml of trifluoroethanol. 30 microliters of acetic acid was added to the resulting solution. Stirring the resulting solution until it is well mixed. Then 12% g/ml by weight of spinning solution was prepared. (3) 0.7 g of gelatin and 0.3 g of polycaprolactone were weighed and dissolved in 10 ml of hexafluoroisopropanol. 40 microliters of acetic acid was added to the resulting solution. Stirring the resulting solution until it was well mixed. Then 10% g/ml by weight of spinning solution was prepared.

Axitinib-loaded gelatin/polycaprolactone nanofiber membranes with a shell-core structure and a sandwich structure were prepared by coaxial electrospinning using the following method: selecting a drum as a receiving device, using the first spinning solution as core spinning solution, using the second spinning solution as shell spinning solution, then performing coaxial electrospinning on 0.8 ml of the two spinning solutions at a given supply rate. Then changing the spinning system. The third spinning solution was separately subjected to electrospinning on the initial drum, the receiving device, for spinning about 1.2 ml of the total volume; finally, using the first spinning solution as core spinning solution, using the second spinning solution as shell spinning solution, and spinning 1 ml of the total volume of the spinning solution by coaxial electrospinning Parameters involved in the electrospinning process were configured as follows: voltage 5-50 kv, receiving distance 5-30 cm, injection rate 0.5-10 ml/h, temperature 10-40° C., relative humidity 20-80%.

### Example 7

Preparation of four different spinning solutions: (1) 300 mg of Axitinib was weighed and dissolved in 5 ml of hexafluoroisopropanol, and the resulting solution was stirred until it was well mixed. (2) 0.4 g of gelatin and 0.8 g of polycaprolactone were weighed and dissolved in 10 ml of trifluoroethanol. 40 microliters of acetic acid was added to the resulting solution. Stirring the resulting solution until it was well mixed. Then 12% g/ml by weight of spinning solution was prepared. (3) 0.5 g of gelatin and 0.5 g of polycaprolactone were weighed and dissolved in 10 ml of trifluoroethanol. 30 microliters of acetic acid was added to the resulting solution. Stirring the resulting solution until it was well mixed. Then 10% g/ml by weight of spinning solution was prepared. (4) 0.7 g of gelatin and 0.5 g of polycaprolactone were weighed and dissolved in 10 ml of hexafluoroisopropanol. 30 microliters of acetic acid was added to the resulting solution. Stirring the resulting solution until it was well mixed. Then 12% g/ml by weight of spinning solution was prepared.

Axitinib-loaded gelatin/polycaprolactone nanofiber membranes with a shell-core structure and a sandwich structure were prepared by coaxial electrospinning using the following method: selecting a drum as a receiving device, using the first spinning solution as core spinning solution, using the second spinning solution as shell spinning solution, then performing coaxial electrospinning on 0.6 ml of the two spinning solutions at a given supply rate. Then changing the spinning system. The third spinning solution was separately subjected to electrospinning on the initial drum, the receiving device, for spinning about 1 ml of the total volume; finally, using the first spinning solution as core spinning solution, using the fourth spinning solution as shell spinning solution, and spinning 1.2 ml of the total volume of the spinning solution by coaxial electrospinning Parameters involved in the electrospinning process were configured as follows: voltage 5-50 kv, receiving distance 5-30 cm, injection rate 0.5-10 ml/h, temperature 10-40° C., relative humidity 20-80%.

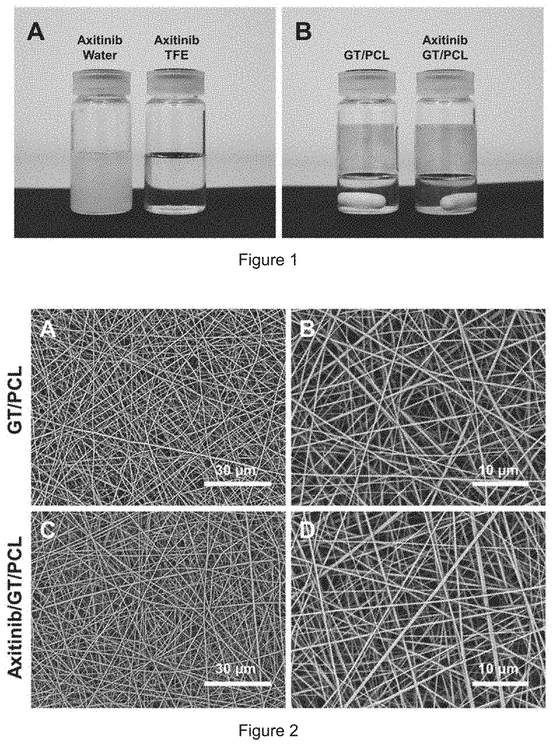

The solubility of Axitinib is shown in FIG. 1. Axitinib is poorly soluble in water. Stirring the solution to forms a milky solution. When it stays for a long period of time, granular precipitation may occur. However, the granular precipitation can be quickly dissolved in trifluoroethanol (TFE) to form a transparent solution (A). 0.25 g of gelatin and 0.25 g of polycaprolactone were dissolved in 5 ml of trifluoroethanol containing 20 μl of acetic acid to form a transparent solution, and 50 mg of Axitinib was added to form a light yellow transparent solution (B).

The scanning electron microscope results were shown in FIG. 2. The nanofiber materials prepared in Example 1 (gelatin/polycaprolactone material) and Example 2 (Axitinib-loaded gelatin/polycaprolactone material) were observed under a scanning electron microscope. It was found that the addition of Axitinib (i.e., a small molecule) did not reduce the spinnability of the material, and the resulting fiber morphology was smooth, uniform, and continuous, and Axitinib could be evenly wrapped in the material without the deposition of drug particles on the fiber surface. In addition, the aperture of the fiber was small, and porosity was high, which can effectively block the contact of the surface of the wound and inflammatory cell infiltration in the body without hindering the delivery and transportation of nutrients and waste.

### Example 8

The heart of a SD rat was separated one day after birth. Cells in the heart tissues (mainly composed of a mixture of cardiomyocytes and fibroblasts) were extracted. The mixed cells were seeded in gelatin/polycaprolactone material (Example 1) and Axitinib-loaded gelatin/polycaprolactone material (Example 2), and cultured for 3 days. Then Live& Dead cells were stained. In addition, human umbilical vein endothelial cell lines were seeded on the two materials and cultured for 1 day, then EdU staining was performed. After the human umbilical vein endothelial cell lines were cultured for 5 days, nuclear staining was performed.

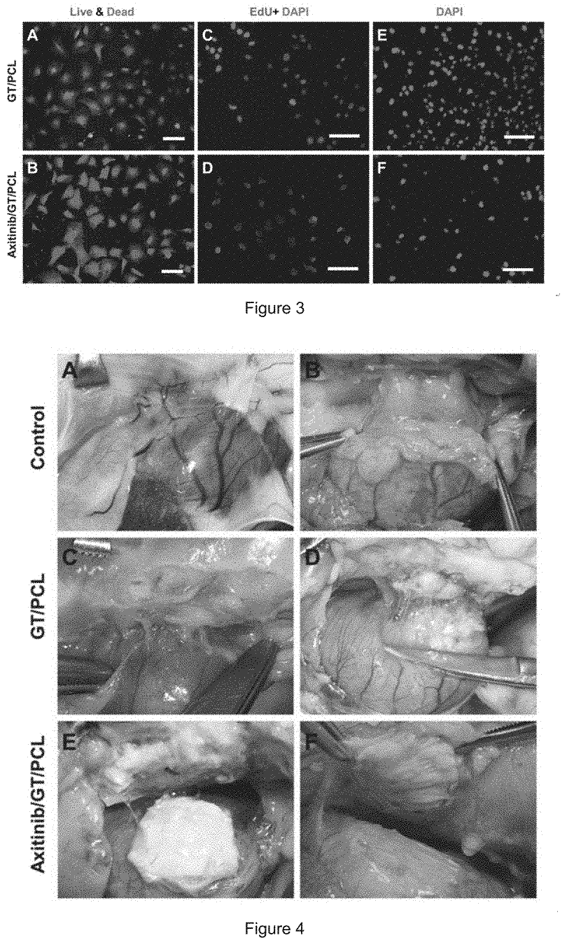

The results were shown in FIG. 3. It can be seen from FIGS. 3A and 3B that the cells in the heart tissues grow well, indicating that the loading of Axitinib has no obvious toxic side effects on the cells in the heart tissues. It can be seen from FIGS. 3C and 3D that a large number of cells on the gelatin/polycaprolactone material are in a proliferation state, and the loading of Axitinib significantly inhibits the proliferation of endothelial cells. It can be seen from FIGS. 3E and 3F that the number of cells on the Axitinib-loaded gelatin/polycaprolactone material is far less than the number of cells on the gelatin/polycaprolactone material, indicating that the loading of Axitinib significantly inhibits the growth of endothelial cells. The above results indicate that the loading of Axitinib does not affect the growth activity of cardiomyocytes and fibroblasts on the materials, but it significantly inhibits the proliferation of the endothelial cells.

### Example 9

Male New Zealand white rabbits (weighing about 2-2.5 kg) were used in the experiment. The rabbits were anesthetized with intravenous injection of 3% sodium pentobarbital (30 mg/kg) at the ear edge, wherein the anesthetization referred to the general anesthetization. The rabbits were placed in a prone position on the operation table for shaving, disinfecting and placing surgical darpe processes. An incision in the skin along the neck was made. Tracheal intubation was performed under the guidance of a guidewire, and intubation tubes were fixed by the expansion of balloons. Turn on a ventilator. If rhythmic contraction and expansion of the thorax appeared, tracheal intubation was successful. Then adjust the ventilator, a small amount of isoflurane was added to the oxygen to maintain deep anesthesia. After intubation and anesthesia were successfully completed, a median sternotomy was done, a sternum retractor was expanded and fixed to separate the thymus and heart fundus tissues, and open the pericardium. An area of about 1.5×1.5 cm of pericardium tissue was cut off. The surface of the heat was wiped several times with sterile dry gauze until punctate hemorrhage occurred. The sterilized material was placed on the defect, four corners were fixed by surgical suture, and the chest was closed as usual. Positive control group: when the surface of the heat was wiped several times with a sterile dry gauze until punctate hemorrhage occurred, no further processes were carried out, and the chest was closed directly. Material groups: the pericardium was repaired with gelatin/polycaprolactone material (Example 1) and Axitinib-loaded gelatin/polycaprolactone material (Example 2), respectively, followed by the closure of the chest. Each rabbit was given an intramuscular injection of cefuroxime (30 mg/kg) to prevent infection before waking up after surgery.

One month after the surgery, open the chest again to carefully separate the adhesion tissues between the sternum and the heart, the implanted material was exposed, and pictures were taken and recorded. In addition, according to related literatures, the adhesion level of each rabbit was scored. Wherein, 0=no adhesion is found between the sternum and the pericardium, and the heart has a clear structure; 1=slight adhesion, but it is easy to perform blunt separation; 2=moderate adhesion, some sharp separation is needed in addition to the blunt separation; 3=severe adhesion, it is easy to bleed during separation, which can be done mainly through sharp separation.

The adhesion level score results are shown in Table 1. The adhesion level between the heart and the sternum of the 6 rabbits in the control group is severe; the adhesion level between the material and the sternum of the 6 rabbits in the gelatin/polycaprolactone material group is severe, wherein for the adhesion between the material and the heart, severe adhesion occurs in 2 rabbits, mediate adhesion occurs in 2 rabbits, and slight adhesion occurs in 2 rabbits. The adhesion level between the material and the sternum of the 6 rabbits in the Axitinib-loaded gelatin/polycaprolactone material group is mediate, wherein for the adhesion between the material and the heart, mediate adhesion occurs in 2 rabbits, and slight adhesion occurs in 4 rabbits. The above-mentioned results show that the adhesion level of animals in the material group is significantly reduced compared with the control group, and the anti-adhesion effect of the material group containing Axitinib (the membrane of Example 2 of the present invention) is the best.

TABLE 1
adhesion level score results in thoracotomy after surgery

| | Adhesion | | | |
|---|---|---|---|---|
| | No adhesion | Slight | Mediate | Severe |
| Groups level | 0point | 1point | 2point | 3point |
| Control group | 0 | 0 | 0 | 6 |
| Gelatin/polycaprolactone | 0 | 0 | 0 | 6 |
| material and the sternum | | | | |
| Gelatin/polycaprolactone | 0 | 2 | 2 | 2 |
| material and the heart | | | | |
| Axitinib-loaded gelatin/ | 0 | 0 | 6 | 0 |
| polycaprolactone material | | | | |
| and the sternum | | | | |
| Axitinib-loaded gelatin/ | 0 | 4 | 2 | 0 |
| polycaprolactone material | | | | |
| and the heart | | | | |

The general view of the sample is shown in FIG. 4: in the positive control group no anti-adhesion material is set, the heart is fully adhered to the sternum; form a large number of fibrous adhesions (FIG. 4A), and the adhesion tissues in the damaged area of the pericardium are dense and firm and cannot be separated (FIG. 4B); in the gelatin/polycaprolactone group (the membrane of Example 1 of the present invention), the adhesion between the material and the sternum is relatively severe, so it is difficult to separate the tissues between the material and the sternum, and a lot of sharp separation is required (FIG. 4 C); and the adhesion between the material and the heart is less severe when compared with the control group. However, the anti-adhesion effect is not very satisfactory. Some sharp separation is still needed to separate adhesions in some areas, which leads to some bleeding on the free surface of the heart, and even occasionally damages myocardial tissues (FIG. 4D); in the Axitinib-loaded gelatin/polycaprolactone group (the membrane in Example 2 of the present invention), mediate and slight adhesions occur between the material and the sternum, and the blunt separation can be used to separate adhesions in the central area of the material (FIG. 4E); only some sharp separation is required at the junction of the material and the autologous pericardial suture. In addition, the adhesion between the material and the heart is mild, and the adhesion is mainly in the form of filamentous adhesion. Thus, the adhesions can be separated simply by the blunt separation. After the material is peeled off, the surface of the heart is smooth and the coronary arteries are clearly visible (FIG. 4F).

The above results show that the Axitinib-loaded gelatin/polycaprolactone nanofiber membrane can not only effectively prevent adhesions between the material and the heart, but also effectively reduce the formation of adhesion between the material and the sternum. The Axitinib-loaded anti-adhesion membrane can greatly reduce the difficulty when the patient undergoes a second cardiac surgery, reduce intraoperative and postoperative bleeding, reduce operation time and costs, and even reduce mortality. In addition, the anti-adhesion membrane is soft, water-permeable, air-permeable, can be cut arbitrarily; moreover, it has advantages of excellent mechanical properties and easy operation; and it is easy to perform surgical suture.

The use of Axitinib-loaded gelatin/polycaprolactone nanofiber membranes with a sandwich structure, a shell-core structure in cardiac surgery has better anti-adhesion effect than Example 1. For example, the sandwich structure allows Axitinib to be located in the inner and outer layers of the material to prevent the material from adhering to the sternum and the heart, while the middle layer of the material does not contain Axitinib, which avoids excessive loading of drugs into the material having side effects on the body and preventing the occurrence of adverse reactions. The “shell-core” structure allows Axitinib to be evenly wrapped in the material, allowing it to be released slowly, so that a sudden release is avoided.

The above descriptions are only the preferred embodiments of the invention, not thus limiting the embodiments and scope of the invention. Those skilled in the art should be able to realize that the schemes obtained from the content of specification and drawings of the invention are within the scope of the invention.

What is claimed is:

1. A use of a nanofiber membrane of small molecule drug in the preparation of medical apparatus for anti-adhesion after a surgery, wherein the small molecule drug, inhibiting vascular endothelial growth factor and/or inhibiting vascular endothelial growth factor receptor, is loaded into the nanofiber membrane.

2. The use of claim 1, wherein the surgery is cardiac surgery.

3. The use of claim 2, wherein the anti-adhesion is to prevent adhesion between the heart to sternum and/or pericardium.

4. The use of claim 1, wherein the small molecule drug is Axitinib.

5. The use of claim 4, wherein the amount of Axitinib loaded in the nanofiber membrane is not less than 1%.

6. The use of claim 5, wherein the amount of Axitinib loaded in the nanofiber membrane is in a range of 2% to 30%.

7. The use of claim 1, wherein the nanofiber membrane is a gelatin/polycaprolactone nanofiber membrane.

8. The use of claim 7, wherein the nanofiber membrane is obtained from a gelatin/polycaprolactone spinning solution containing Axitinib by electrospinning.

9. The use of claim 7, wherein the nanofiber membrane is produced by a method comprising the steps of: preparing a first spinning solution and a second spinning solution, respectively, wherein the first spinning solution is gelatin/polycaprolactone spinning solution containing Axitinib, and the second spinning solution is gelatin/polycaprolactone spinning solution not containing Axitinib; extracting the first spinning solution, the second spinning solution in sequence, and the first spinning solution is implemented for electrospinning process; finally, obtaining a nanofiber membrane with a sandwich structure.

10. The use of claim 7, wherein the nanofiber membrane is produced by a method comprising the steps of: preparing a core spinning solution and a shell spinning solution, respectively, wherein the core spinning solution contains Axitinib, and the shell spinning solution is gelatin/polycaprolactone spinning solution not containing Axitinib; performing coaxial electrospinning on the two spinning solution; and finally, obtaining a nanofiber membrane with a shell-core structure.
